(19) United States
(12) Patent Application Publication
(10) Pub. No.: US 2026/0188463 A1
(43) Pub. Date: Jul. 2, 2026

(54) PERSONAL DIETARY SUPPLEMENT DISPENSER AND METHODS FOR PREPARING PERSONALIZED DIETARY SUPPLEMENTS

(71) Applicant: Agen Com Pte. Ltd., SINGAPORE (SG)

(72) Inventors: Vladimir SITNIKOV, Singapore (SG); Alexander TORMASOV, Busingen am Hochrhein (DE); Serg BELL, Singapore (SG)

(21) Appl. No.: 19/003,472

(22) Filed: Dec. 27, 2024

(51) Int. Cl. G16H 20/60; H04W 4/80

(52) U.S. Cl. CPC G16H 20/60; H04W 4/80

(57) ABSTRACT

The present disclosure relates to a personal dietary supplement dispenser and methods for preparing personalized dietary supplements. An example personal dietary supplement dispenser comprises a one or more containers, each configured to store one or more dietary supplement ingredients; one or more reservoirs, each configured to store one or more liquids, powders, or gels; a controller, configured to control operation of the personal dietary supplement dispenser, and generate a personalized dietary supplement for a user, by causing the personal dietary supplement dispenser to combine and/or mix one or more portions of the one or more dietary supplement ingredients, optionally using one or more portions of the one or more liquids, powders, or gels, in response to receiving a recipe for the personalized dietary supplement from the server; and transmit information about the source, type, status, quantity, and/or amount of the one or more dietary supplement ingredients to the server.

### TECHNICAL FIELD

The present disclosure relates to a personalized dietary supplements, and devices and systems configured to produce the same using machine learning and/or other parameters, techniques and algorithms set forth here.

### BACKGROUND

In the U.S. and elsewhere, consumers are increasingly turning to dietary supplements (e.g., containing vitamins, minerals, probiotics, and other functional components) to improve their health and well-being, and to help achieve personal goals such as weight loss. Consumers typically obtain individual, pre-packaged dietary supplements and simply ingest a recommended amount daily, or according to a generic dosage schedule set by the manufacturer or recommended by a medical professional. Some personalized solutions have been proposed and/or commercialized. However, these systems typically lack extensive customization options and are not designed to account for the combination or mixture of multiple ingredients. Moreover, these systems typically consist of nothing more than a regimen for the user to follow by manually preparing recommended amounts of multiple ingredients. As such, prior systems do not often truly personalized dietary supplements, nor do they provide a simple and efficient system for preparing personalized dietary supplements.

### BRIEF DESCRIPTION

The present disclosure provides systems, devices, and methods for preparing personalized dietary supplements which offer various benefits and advantages compared to known options in this area. For example, the present disclosure provides devices for preparing a personalized dietary supplement that may be conveniently stored in a user’s kitchen (or elsewhere in a home), and which store and track inventory of various dietary supplement components as part of a single, convenient, integrated system. Such devices may be configured to allow a user to manually design a personalized dietary supplement recipe, or to select and/or customize a personalized dietary supplement recipe (e.g., based on existing recipes stored in memory). In some aspects, the devices described herein may be configured to communicate with a remote server or one or more other local or remote databases or other sources of information. For example, the device may obtain personalized dietary supplement recipes from a remote server configured to design new recipes, or to customize existing recipes, for a user based on machine learning techniques or other algorithms, parameters. In some aspects, the disclosure provides a system that includes a remote server configured to generate personalized dietary supplement recipes for users based on health and/or fitness data for the user, and/or various other parameters described herein, and a device configured to communicate with the server and to prepare a personalized dietary supplement using these recipes, providing a convenient solution for consumers looking to improve their health, diet, or to achieve other personal goals using dietary supplements.

In a first general aspect, the disclosure provides a personal dietary supplement dispenser, comprising a one or more containers, each configured to store one or more dietary supplement ingredients; one or more reservoirs, each configured to store one or more liquids, powders, or gels; a communication system; a controller, configured to control operation of the personal dietary supplement dispenser; wherein the controller is further configured to communicate with at least one remote server using the communication system, generate a personalized dietary supplement for a user, by causing the personal dietary supplement dispenser to combine and/or mix one or more portions of the one or more dietary supplement ingredients, optionally using one or more portions of the one or more liquids, powders, or gels, in response to receiving a recipe for the personalized dietary supplement from the server; and transmit information about the source, type, status, quantity, and/or amount of the one or more dietary supplement ingredients to the server.

In some aspects, the personal dietary supplement dispenser further comprises: a user interface configured to allow the user to (a) display a recipe generated or received by the personal dietary supplement dispenser, to the user; (b) manually create a recipe for a personalized dietary supplement to prepare; and/or (c) modify one or more parameters of the personalized dietary supplement recipe received from the server

In some aspects, the personal dietary supplement dispenser further comprises: a) one or more heating or cooling elements, wherein the controller is further configured to heat or cool the personalized dietary supplement using the one or more heating or cooling elements; b) one or more mixing elements, wherein the controller is further configured to mix one or more of the dietary supplement ingredients and/or the personalized dietary supplement, using the one or more mixing elements; and/or c) one or more sensors, each capable of detecting one or more signals indicative or a viscosity, temperature, and/or other parameter(s) of the one or more dietary supplement ingredients and/or the personalized dietary supplement.

In some aspects, the controller is further configured to collect and store usage data for the one or more dietary supplement ingredients and/or the stored liquids, powders, or gels, using one or more sensors, and transmit the usage data to the remote server using the communication system.

In some aspects, each of the one or more containers includes a machine-readable label comprising information about a source, type, quantity, amount, and/or expiration date, of the dietary supplement ingredient contained in each respective container; and the controller is configured to cause the personal dietary supplement dispenser to read the machine-readable label using one or more sensors.

In some aspects, the controller is configured to detect when a new container containing a dietary supplement ingredient is added to the one or more containers, and to validate the new container by reading the machine-readable label associated with the new container, and transmit information based on the machine-readable label to the server.

In some aspects, the controller is configured to cause the personal dietary supplement dispenser to use the dietary supplement ingredient stored in the new container only after the new container has been validated.

In some aspects, the controller is further configured to wirelessly communicate with one or more mobile or wearable electronic devices and to allow a user to manually select, create, or modify a personalized dietary supplement recipe using the mobile or wearable electronic device.

In some aspects, the controller is further configured to generate personalized dietary supplements according to a preset schedule and/or based on user proximity data.

In some aspects, the controller is further configured to contact the remote server according to a preset schedule and to download one or more personalized dietary supplement recipes for the user to a local memory of the personal dietary supplement dispenser.

In some aspects, the communication system is a wireless communication system, optionally one that utilizes a near-field communication (NFC) protocol, Bluetooth, or another protocol.

In some aspects, the personal dietary supplement dispenser further comprises one or more sensors, each configured to detect and/or measure at least one parameter of the stored dietary supplement ingredients and/or the stored liquids, powders, or gels.

In some aspects, the server is a smartphone, computer, electronic device, tablet, a remote server, or a cloud-based computing platform.

In a second general aspect, the disclosure provides a method for generating a personalized dietary supplement for a user, comprising: a) providing a personal dietary supplement dispenser comprising one or more containers, each configured to store one or more dietary supplement ingredients; one or more reservoirs, each configured to store one or more liquids, powders, or gels; a communication system; and a controller, configured to control operation of the personal dietary supplement dispenser and to communicate with at least one server using the communication system; b) receiving, by the controller, a personalized dietary supplement recipe for the user, from the server; and c) generating a personalized dietary supplement for the user, using the personal dietary supplement dispenser, by combining and/or mixing one or more portions of the one or more dietary supplement ingredients, optionally using one or more portions of the one or more liquids, powders, or gels, based on the received personalized dietary supplement recipe.

In some aspects, the method further comprises: transmitting information about the source, type, status, quantity, amount, and/or expiration date, of the one or more dietary supplement ingredients, from the personal dietary supplement dispenser to the server before or after generating the personalized dietary supplement for the user.

In some aspects, the method further comprises: receiving input from the user, via a user interface of the personal dietary supplement dispenser, wherein the user input modifies one or more parameters of the personalized dietary supplement recipe received from the server

In some aspects, the method further comprises: a) heating or cooling the personalized dietary supplement using a heating or cooling element integrated into the personal dietary supplement dispenser; b) mixing one or more of the dietary supplement ingredients and/or the personalized dietary supplement, using one or more mixing elements integrated into the personal dietary supplement dispenser; and/or c) detecting one or more signals indicative or a viscosity, temperature, and/or other parameter(s) of the one or more dietary supplement ingredients and/or the personalized dietary supplement, using one or more sensors integrated into the personal dietary supplement dispenser.

In some aspects of the methods described herein, the controller is further configured to collect and store usage data for the one or more dietary supplement ingredients and/or the stored liquids, powders, or gels, using one or more sensors, and transmit the usage data to the server using the communication system.

In some aspects of the methods described herein, each of the one or more containers includes a machine-readable label comprising information about the source, type, quantity, amount, or expiration date, of the dietary supplement ingredients contained in each respective container; and the controller is configured to cause the personal dietary supplement dispenser to read the machine-readable label using one or more sensors.

In some aspects of the methods described herein, the controller is configured to detect when a new container containing a dietary supplement ingredient is added to the one or more containers, and to validate the new container by reading the machine-readable label associated with the new container, and transmit information based on the machine-readable label to the server.

In some aspects of the methods described herein, the controller is further configured to communicate with one or more mobile or wearable electronic devices and to allow a user to manually select, create, or modify a personalized dietary supplement recipe using the mobile or wearable electronic device.

In some aspects of the methods described herein, the controller is further configured to generate personalized dietary supplements according to a preset schedule and/or based on user proximity data.

In some aspects of the methods described herein, the controller is further configured to communicate with the server, optionally according to a preset schedule, and to download one or more personalized dietary supplement recipes for the user to a local memory of the personal dietary supplement dispenser.

In some aspects of the methods described herein, the communication system is a wireless communication system, optionally one that utilizes a near-field communication (NFC) protocol, Bluetooth, or another protocol.

In some aspects of the methods described herein, the personal dietary supplement dispenser further comprises one or more sensors, each configured to detect and/or measure at least one parameter of the stored dietary supplement ingredients and/or the stored liquids, powders, or gels.

In some aspects of the methods described herein, the server is a smartphone, computer, electronic device, tablet, a remote server, or a cloud-based computing platform.

In a third general aspect, the disclosure provides a method for generating a personalized dietary supplement for a user of a dietary supplement dispenser, comprising: receiving, by a server, health and/or fitness data for the user comprising one or more of (a) biometric data obtained from one or more wearable electronic devices, (b) medical test data, (c) genetic test data, and (d) health and/or fitness information; receiving, by the server, dietary supplement component data comprising information about one or more dietary supplement components; training, by the server, one or more machine learning (ML) models, for assessing a health and/or fitness state of the user using (i) the received health and/or fitness data for the user, and/or health and/or fitness data obtained from a population of other human subjects, and (ii) the received dietary supplement component data; receiving, by the server, (i) input from the user comprising one or more parameters for the personalized dietary supplement, obtained via an interface of an application executed on a mobile device or a computer of the user, and/or (ii) current health and/or fitness data for the user; generating a recipe for the personalized dietary supplement, by the server, by customizing an existing recipe for the user, optionally wherein customization is based on availability of one or more dietary supplement components in the dietary supplement dispenser, and/or a target minimum quantity, amount, or volume of the one or more dietary supplement components to be consumed by the user within a predetermined time period; and/or using the one or more trained ML models, and (i) the received one or more parameters for the personalized dietary supplement, and/or (ii) the current health and/or fitness data for the user; transmitting the recipe, from the server to a dietary supplement dispenser configured to prepare the personalized dietary supplement using the recipe.

In some aspects, the server is further configured to generate the recipe for the personalized dietary supplement using supplemental health and/or fitness data for the human subject comprising one or more of (a) input provided by a medical practitioner, (b) prior or concurrent drug consumption information, (c) prior or concurrent dietary supplement consumption information, (d) health and/or fitness information about the human subject obtained from a third-party application, (e) health and/or fitness information about the human subject obtained from a third-party database; (f) information describing a quantity, amount, concentration of the one or more dietary supplement components in the dietary supplement dispenser, or availability of one or more dietary supplement components available for delivery to the user; and/or (g) information regarding minimum recommended and/or maximum allowed dosage, quantity, amount, or volume of the one or more dietary supplement components.

In some aspects, the one or more ML models comprise one or more pre-trained models that have been trained using a dataset comprising health and/or fitness data obtained from a plurality of users, wherein the dataset comprises (a) biometric data obtained from one or more wearable electronic devices, (b) medical test data, (c) genetic test data, and/or (d) health and/or fitness information, obtained from or associated with the plurality of users.

In some aspects, the server is further configured to receive, from the dietary supplement dispenser, information describing one or more dietary supplement components available in the dietary supplement dispenser, and optionally a quantity, amount and/or concentration of each of the one or more dietary supplement components.

In some aspects, the server is further configured to generate the recipe for the personalized dietary supplement using one or more pre-trained ML models, each comprising an ML model that has been trained using information comprising nutritional information and/or functional properties of one or more dietary supplement components.

In some aspects, the server is further configured to generate the recipe for the personalized dietary supplement by generating a plurality of potential dietary supplement recipes using the one or more pre-trained ML models, based on (i) the received one or more parameters for the personalized dietary supplement, and/or (ii) the current health and/or fitness data for the user; and scoring the potential dietary supplement recipes; and selecting a highest-scoring potential dietary supplement recipe as the personalized dietary supplement for the user.

In some aspects, the server is further configured to score the potential dietary supplement recipes using a scoring algorithm that accounts for the one or more health and/or fitness goals of the user, and assigns a higher score to potential dietary supplement recipes that are determined to be more likely to result in the user achieving the one or more health and/or fitness goals.

In some aspects, the server is further configured to score the potential dietary supplement recipes using a scoring algorithm that accounts for nutritional and/or functional properties of the one or more dietary supplement components included in each potential dietary supplement recipe.

In some aspects, the potential dietary supplement recipes are scored based on the input from the human subject comprising one or more parameters for the personalized dietary supplement.

In some aspects, the one or more parameters for the personalized dietary supplement comprise: a) a selection of one or more dietary supplement components that must be included in the personalized dietary supplement; b) a selection of one or more dietary supplement components that must not be included in the personalized dietary supplement; c) an order of addition and/or mixing of one or more dietary supplement components; d) a time to begin preparation of the personalized dietary supplement; and/or e) a temperature and/or size of the personalized dietary supplement.

In some aspects, the one or more health and/or fitness goals of the user comprise: (a) an improvement in cardiovascular health, (b) an improvement in muscle development, and/or (c) a goal related to weight gain or loss, metabolism, mood improvement, stress reduction, reduced inflammation, improved immune system functioning, improved sleep, one or more hormone levels, antioxidant consumption, cognition, energy, digestive health, improved, liver functioning, gut microbiome composition, liver health, bone health, cell maintenance, and/or an improvement to the skin, hear or nails of the user.

In some aspects, the health and/or fitness data for the user received by the server further comprises: (e) information describing one or more health and/or fitness goals of the user.

In some aspects, the server comprises a computer, a smartphone, a tablet device, or a distributed computing platform.

In some aspects, the availability of one or more dietary supplement components in the dietary supplement dispenser is based on whether the one or more dietary supplement components are: a) in stock in the device, b) available from a supplier, c) available for delivery within a defined period of time, or d) any combination of a) to c).

In a fourth general aspect, the disclosure provides a system for generating a personalized dietary supplement for a human subject, comprising: a server, configured to receive health and/or fitness data for the user comprising one or more of (a) biometric data obtained from one or more wearable electronic devices, (b) medical test data, (c) genetic test data, and (d) health and/or fitness information; receive dietary supplement component data comprising information about one or more dietary supplement components; train one or more machine learning (ML) models, for assessing a health and/or fitness state of the user using (i) the received health and/or fitness data of the user, and/or health and/or fitness data obtained from a population comprising other human subjects, and (ii) the received dietary supplement component data; receive (i) input from the user comprising one or more parameters for the personalized dietary supplement, obtained via an interface of an application executed on a mobile device or a computer of the user, and/or (ii) current health and/or fitness data for the user; generate a recipe for the personalized dietary supplement (a) by customizing an existing recipe for the user, optionally wherein customization is based on availability of one or more dietary supplement components in the dietary supplement dispenser, and/or a target minimum quantity, amount or volume of the one or more dietary supplement components to be consumed by the user within a predetermined time period; and/or (b) using the one or more trained ML models, and (i) the received one or more parameters for the personalized dietary supplement, and/or (ii) the current health and/or fitness data for the user; transmit the recipe to a dietary supplement dispenser configured to prepare the personalized dietary supplement using the recipe.

In some aspects, the system comprises a server that is further configured to generate the recipe for the personalized dietary supplement using supplemental health and/or fitness data for the human subject comprising one or more of (a) input provided by a medical practitioner, (b) prior or concurrent drug consumption information, (c) prior or concurrent dietary supplement consumption information, (d) health and/or fitness information about the human subject obtained from a third-party application, and (e) health and/or fitness information about the human subject obtained from a third-party database.

In some aspects, the system comprises one or more ML models that each comprise one or more pre-trained models that have been trained using a dataset comprising health and/or fitness data obtained from a plurality of users, wherein the dataset comprises (a) biometric data obtained from one or more wearable electronic devices, (b) medical test data, (c) genetic test data, and/or (d) health and/or fitness information, obtained from or associated with the plurality of users.

In some aspects, the system comprises a server that is further configured to receive, from the dietary supplement dispenser, information describing one or more dietary supplement components available in the dietary supplement dispenser, and optionally an amount and/or concentration of each of the one or more dietary supplement components.

In some aspects, the system comprises a server that is further configured to generate the recipe for the personalized dietary supplement using (a) one or more pre-trained ML models, each comprising an ML model that has been trained using information comprising nutritional information and/or functional properties of one or more dietary supplement components; and/or (b) one or more predetermined instructions or limitations.

In some aspects, the system comprises a server that is further configured to generate the recipe for the personalized dietary supplement by generating a plurality of potential dietary supplement recipes using the one or more pre-trained ML models, based on (i) the received one or more parameters for the personalized dietary supplement, and/or (ii) the current health and/or fitness data for the user; and scoring the potential dietary supplement recipes; and selecting a highest-scoring potential dietary supplement recipe as the personalized dietary supplement for the user.

In some aspects, the system comprises a server that is further configured to score the potential dietary supplement recipes using a scoring algorithm that accounts for the one or more health and/or fitness goals of the user, and assigns a higher score to potential dietary supplement recipes that are determined to be more likely to result in the user achieving the one or more health and/or fitness goals.

In some aspects, the system comprises a server that is further configured to score the potential dietary supplement recipes using a scoring algorithm that accounts for nutritional and/or functional properties of the one or more dietary supplement components included in each potential dietary supplement recipe.

In some aspects, the system comprises a server that is further configured to score the potential dietary supplement recipes based on the input from the human subject comprising one or more parameters for the personalized dietary supplement.

In some aspects of the systems described herein, the one or more parameters for the personalized dietary supplement comprise: a) a selection of one or more dietary supplement components that must be included in the personalized dietary supplement; b) a selection of one or more dietary supplement components that must not be included in the personalized dietary supplement; and/or c) a temperature and/or size of the personalized dietary supplement.

In some aspects of the systems described herein, the one or more health and/or fitness goals of the user comprise (a) an improvement in cardiovascular health, (b) an improvement in muscle development, and/or (c) a goal related to weight gain or loss, metabolism, mood improvement, stress reduction, reduced inflammation, improved immune system functioning, improved sleep, one or more hormone levels, antioxidant consumption, cognition, energy, digestive health, improved, liver functioning, gut microbiome composition, liver health, bone health, cell maintenance, and/or an improvement to the skin, hear or nails of the user.

It should be noted that the methods described above may be implemented in a system comprising a hardware processor. Alternatively, the methods may be implemented using computer executable instructions of a non-transitory computer readable medium.

The above simplified summary of example aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is block diagram showing aspects of an exemplary system for generating a personalized dietary supplement according to the present disclosure. In this case, the system includes a mobile device 101, one or more remote servers 102, a supplement dispenser 103, and several sources of data and/or parameters that ma be used to generate personalized supplements, 104-107.

FIG. 2 is a block diagram showing aspects of an exemplary system for generating a personalized dietary supplement according to the present disclosure. This figure highlights potential data sources and the layout of modules used in some aspects of the systems described herein.

FIG. 3 is a block diagram showing aspects of an exemplary system for generating a personalized dietary supplement according to the present disclosure. This figure highlights potential data sources and the layout of modules used in some aspects of the systems described herein.

FIG. 4 is a flow chart showing an exemplary method for generating a personalized dietary supplement according to the present disclosure.

FIG. 5 is a flow chart showing another exemplary method for generating a personalized dietary supplement according to the present disclosure.

FIG. 6 is a flow chart showing an exemplary method for generating a personalized dietary supplement according to the present disclosure.

FIG. 7 is a block diagram illustrating a computer system 20 on which aspects of systems and methods for generating custom courses on a user interface using machine learning may be implemented in accordance with an exemplary aspect.

### DETAILED DESCRIPTION

Exemplary aspects are described herein in the context of systems and methods for generating personalized dietary supplements using machine learning, predetermined rules and/or parameters, and/or user input, as well as other sources of input and/or data described herein. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the example aspects as illustrated in the accompanying drawings. The same reference indicators will be used to the extent possible throughout the drawings and the following description to refer to the same or like items.

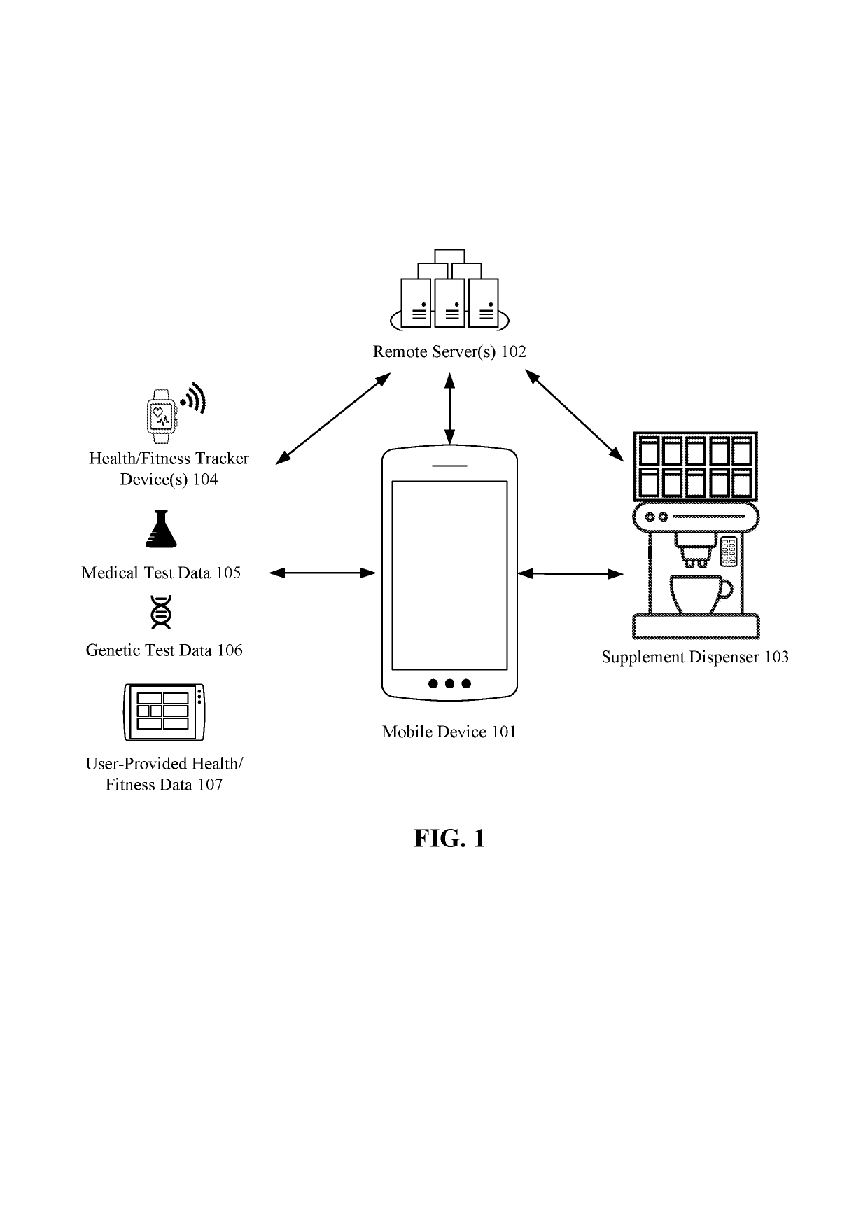

FIG. 1 is block diagram showing aspects of an exemplary system for generating a personalized dietary supplement according to the present disclosure. In this case, the system 100 includes a mobile device 101, one or more remote servers 102, a supplement dispenser 103, and several sources of data and/or parameters that may be used to generate personalized supplements, 104-107.

As illustrated by this figure, a user may be able to interact with the supplement dispenser 103 and/or the other components of the system 100 using a mobile device 101. In this example, the mobile device 101 is a user’s smart phone. In other embodiments, the mobile device 101 may comprise a wearable device (e.g., a smart watch), a tablet, a laptop, or a dedicated controller for the device. It is understood that in some embodiments the user may be able to interact with the components of the system 100 using a device that it not typically mobile (e.g., a desktop computer). Thus, any references to systems that utilize a mobile device 100 should be understood as also contemplating alternatives using fixed or non-mobile devices as well, in alternative embodiments. Regardless, the mobile device 101 (or other means of control) may be configured to execute software code designed to generate a graphical user interface (GUI) that allows the user to interact with the supplement dispenser 103. For example, a GUI may allow a user to view the status of the supplement dispenser 103, and/or the current quantity, amount, concentration, and/or volume of one or more dietary supplement components stored in the supplement dispenser 103. In some aspects, the GUI may allow a user to view, select, and/or customize dietary supplement recipes stored on the supplement dispenser 103 or on a remote server 102 (e.g., a database of recipes provided by the manufacturer of the supplement dispenser 103 or third-party databases). For example, the GUI may allow a user to select a dietary supplement recipe stored in memory of the supplement dispenser 103 and to modify the recipe my selecting one or more dietary supplement components to add or remove, or by adjusting the amount, quantity, concentration, or volume of one or more dietary supplement components.

In some aspects, the dietary supplement components may be stored in capsules or containers having one or more human and/or machine-readable labels, tags, or chips. For example, a container or capsule may comprise a near-field communication (NFC) or other wirelessly readable tag that stores information about the manufacturer of a dietary supplement component, or production-related information (e.g., series, date of manufacture, expiration date, etc.) or a unique identifier. The personal dietary supplement dispenser may be configured to wireless read this NFC (or other wirelessly-readable tag) in order to validate the authenticity of dietary supplement components, and optionally to block the use of counterfeit, unidentified, and or non-validated dietary supplement components. In some aspects, the human and/or machine-readable labels, tags, or chips may describe information about the amount, level, or concentration of the dietary supplement component in a container or capsule. Other information may also be described, e.g., the time since first (or a most recent) use of the component may be recorded, and the storage temperature of the component may be tracked. It is envisioned that in some aspects, the human and/or machine-readable labels, tags, or chips may be editable (e.g., by the dietary supplement dispenser, or by an app or device (e.g., by an app run on a mobile device of the user, or via a computer or electronic device operated by the user). For example, the personal dietary supplement dispenser may be allowed to read a parameter indicative of an initial amount, level, or concentration of a component, and to record to the label, tag, or chip a new amount, level, or concentration after using a portion of that component in a formula. The reading and writing process may be performed using, e.g., a wireless means of communication such as NFC or Bluetooth. In some aspects, a personal dietary supplement dispenser may comprise 1, 2, 3, 4, or 5 NFC readers (or reader/writer elements) positioned at one or more locations in or on the device. It is further envisioned that the personal dietary supplement dispenser may include one or more NFC readers that allow a user to authorize the generation of a personalized dietary supplement via a one-touch process. For example, a user may be able to authorize the device to proceed with preparing a personalized dietary supplement by tapping an NFC reader interface on the device with a smartphone, key fob, card, or other NFC-compatible device.

In some aspects, the supplement dispenser 103 may receive dietary supplement recipes from a remote server 102 that has been configured to generate dietary supplement recipes for a user. As shown by FIG. 1, the remote server 102 may communicate with various data sources, including without limitation health/fitness tracker devices 104, medical test data 105 sources, genetic test data 106 sources, and user-provided health/fitness data 107. These data sources may comprise, e.g., one or more third-party database, one or more internal databases maintained by the operator of the remote server 102 or the manufacturer of the supplement dispenser 103, or real-time or historic data stored or generated by one or more devices or sensors (e.g., wearable fitness trackers). The remote server 102 may be configured to generate personalized dietary supplement recipes using any combination of these data sources (or any other data sources described herein), using one or more algorithms, predetermined or user-selected parameters, and/or machine learning techniques.

In some aspects, the remote server 102 may store a library of existing recipes, e.g., collected from books or the internet (e.g., webpages run by “influencers” and other online personalities), or designed by medical or health professionals, and generate a personalized dietary supplement recipe for a given user by customizing one of these existing templates. For example, a user may use the GUI of the mobile device 101 to indicate that they desire to achieve a particular goal (e.g., weight loss), or to increase their consumption of a given dietary supplement component, or to indicate that a given dietary supplement should never be included in a recipe (e.g., due to personal preferences or allergies). The remote server 102 may take any such parameters into account and modify an existing recipe template accordingly.

Similarly, the remote server 102 may take into account health/fitness tracker device 104 data (e.g., real-time or historical data regarding one or more physiological or health-related parameters, or activity-related data, for the user). For example, data from a health/fitness tracker device 104 may comprise data from a smart watch or other wearable device, and may show that a user is engaged in a rigorous physical activity or exercise. In that case, the remote server 102 may account for this data by, e.g., increasing the calories or providing additional electrolytes, in a dietary supplement recipe for the user. Medical test data 105 and genetic test data 106 may also be utilized. For example, medical test data 105 may comprise the results of a recent diagnostic panel showing that the user is deficient in a given nutrient or vitamin (e.g., the user may have low iron levels). The remote server 102 may account for this data by, e.g., by adding iron or increasing the concentration of iron in a dietary supplement recipe for the user. Genetic test data 106 may provide similar insights, e.g., such data may reveal that a user has a genetic signature associated with a given health and/or fitness state or outcome, which the remote sever 102 can account for when designing and/or modifying dietary supplement recipes for that user. For example, a user may have a gene that results in slow metabolism, reduced immune system functionality, or another negative characteristic, which can be corrected or mitigated by consumption of a particular dietary supplement component.

In some aspects, the remote server 102 may be configured to account for user-provided health and/or fitness data 107. Such data may be obtained from the user, e.g., via a questionnaire provided in the GUI of an application executed on the mobile device 101, or otherwise made available to the user. For example, the GUI may allow a user to input historical health and/or fitness data (e.g., information about the user’s health, weight, activity level, goals). The remote server 102 may account for this data when formulating or modifying dietary supplement recipes.

In some aspects, the remote server 102 may generate a personalized dietary supplement recipe for a user using one or more algorithms, statistical techniques, or using artificial intelligence (AI) or machine learning (ML) models. For example, an AI/ML model for assessing a health and/or fitness state of the user may be trained using biometric, dietary, health and/or fitness data obtained from or associated with the user, and optionally using historical data from other users. Such models may be trained, e.g., to recognize deficiencies in a user’s diet that can be corrected using a personalized dietary supplement, or dietary supplement components that should be added to a user’s personalized dietary supplement in order to help the user achieve a desired goal (e.g., weight loss, muscle development, improved cardiovascular health, or any other goal described herein). ML models may also be used to design or modify a dietary supplement recipe for the user. For example, the remote server 102 may be configured to generate a set of candidate dietary supplement recipes for a user, and the set can be scored using statistical techniques, or AI/ML algorithms or models in order to identify the best recipe for a user. The best recipe may comprise, e.g., a recipe that is determined based on a scoring metric to be most likely to (a) result in a desired health or fitness outcome or goal selected by the user (e.g., weight loss), or (b) improve the state of the user’s health and/or fitness with respect to one or more physiological parameters or activities (e.g., an improvement to the quality of the user’s sleep or metabolism). For example, candidate recipes may be scored using a model that was previously trained using historical biometric, dietary, health and/or fitness data for a population of users (e.g., where the trained model recognizes. In this case, the pre-trained model may be used to recognize correlations between the historical biometric, health and/or fitness data, and dietary supplement consumption data, for the users in that population. The trained model may reveal, e.g., that users that consume a higher quantity, amount, concentration, or volume or a given dietary supplement component consistently experienced a particular change in one or more physiological parameters, or a particular health and/or fitness outcome (e.g., a pattern may become evident showing that regular consumption of a given vitamin results in a favorable reduction in blood pressure, or a tendency for a user to experience weight loss, muscle gain, or another positive health and/or fitness outcome described herein).

Note that while the foregoing examples contemplate that the remote server 102 may be configured to perform AI/ML-related analysis, in alternative aspects this functionality may be performed by the supplement dispenser 103. The decision to have this functionality performed locally on device or remotely may vary among different implementations.

Various statistical techniques, algorithms, and AI/ML models may be implemented by the systems and devices described herein, when generating or scoring dietary supplement recipes. A non-limiting set of examples is provided below.

In some aspects, the algorithm may comprise a linear regression algorithm. Linear regression algorithms are disclosed in James, Witten, Hastie, and Tibshirani, An Introduction to Statistical Learning, 2013, Springer Science + Business Media New York, which is hereby incorporated by reference. In some aspects, the algorithm may comprise a logistic regression algorithm. Logistic regression algorithms are disclosed in Agresti, An Introduction to Categorical Data Analysis, 1996, Chapter 5, pp. 103-144, John Wiley & Son, New York, N.Y., which is hereby incorporated by reference. In some aspects, the algorithm may comprise a linear discriminant analysis algorithm. Linear discriminant analysis algorithms are as disclosed in Izenman, 2013, “Linear Discriminant Analysis,” In: Modern Multivariate Statistical Techniques, Springer Texts in Statistics. Springer, New York, N.Y., which is hereby incorporated by reference.

The AI/ML models used herein may be implemented using computer executable software, firmware, hardware, or combinations thereof. For example, these AI/ML models may include reference to a processor and supporting data storage. Further, the AI/ML models may be implemented across multiple devices or other components local or remote to one another. These AI/ML models may be implemented in a centralized system, or as a distributed system for additional scalability. Moreover, any reference to software may include non-transitory computer readable media that when executed on a computer, causes the computer to perform one or more steps.

There are many potential AI/ML models that can be used by the systems and methods described herein. Machine and deep learning classifiers include but are not limited to AdaBoost, Artificial Neural Network (ANN) learning algorithm, Bayesian belief networks, Bayesian classifiers, Bayesian neural networks, Boosted trees, case-based reasoning, classification trees, Convolutional Neural Networks, decisions trees, Deep Learning, elastic nets, Fully Convolutional Networks (FCN), genetic algorithms, gradient boosting trees, k-nearest neighbor classifiers, LASSO, Linear Classifiers, naive Bayes classifiers, neural nets, penalized logistic regression, Random Forests, ridge regression, support vector machines, or an ensemble thereof. See, e.g., Han & Kamber (2006) Chapter 6, Data Mining, Concepts and Techniques, 2nd Ed. Elsevier: Amsterdam. As described herein, any classifier or combination of classifiers (e.g., an ensemble) may be used by the present systems.

### Deep Learning Algorithms

In some aspects, the classifier is a deep learning algorithm. Machine learning is a subset of artificial intelligence that uses a machine’s ability to take a set of data and learn about the information it is processing by changing the algorithm as data is being processed. Deep learning is a subset of machine learning that utilizes artificial neural networks inspired by the workings on the human brain. For example, the deep learning architecture may be multilayer perceptron neural network (MLPNN), backpropagation, Convolutional Neural Network (CNN), Recurrent Neural Network (RNN), Long Short-Term Memory (LSTM), Generative Adversarial Network (GAN), Restricted Boltzmann Machine (RBM), Deep Belief Network (DBN), or an ensemble thereof.

### Classification Trees

A classification tree is an easily interpretable classifier with built in feature selection. A classification tree recursively splits the data space in such a way so as to maximize the proportion of observations from one class in each subspace.

The process of recursively splitting the data space creates a binary tree with a condition that is tested at each vertex. A new observation is classified by following the branches of the tree until a leaf is reached. At each leaf, a probability is assigned to the observation that it belongs to a given class. The class with the highest probability is the one to which the new observation is classified. Classification trees are essentially a decision tree whose attributes are framed in the language of statistics. They are highly flexible but very noisy (the variance of the error is large compared to other methods).

Tools for implementing classification tree are available, by way of non-limiting example, for the statistical software computing language and environment, R. For example, the R package “tree,” version 1.0-28, includes tools for creating, processing and utilizing classification trees. Examples of Classification Trees include but are not limited to Random Forest. See also Kamiński et al. (2017) “A framework for sensitivity analysis of decision trees.” Central European Journal of Operations Research. 26(1): 135-159; Karimi & Hamilton (2011) “Generation and Interpretation of Temporal Decision Rules”, International Journal of Computer Information Systems and Industrial Management Applications, Volume 3, the content of which is incorporated by reference in its entirety.

### Random Forest Classifiers

Classification trees are typically noisy. Random forests attempt to reduce this noise by taking the average of many trees. The result is a classifier whose error has reduced variance compared to a classification tree. Methods of building a Random Forest classifier, including software, are known in the art. Prinzie & Poel (2007) “Random Multiclass Classification: Generalizing Random Forests to Random MNL and Random NB.” Database and Expert Systems Applications. Lecture Notes in Computer Science. 4653; Denisko & Hoffman (2018) “Classification and interaction in random forests.” PNAS 115(8): 1690-1692, the contents of which are incorporated by reference in its entirety.

To classify a new observation using the random forest, classify the new observation using each classification tree in the random forest. The class to which the new observation is classified most often amongst the classification trees is the class to which the random forest classifies the new observation. Random forests reduce many of the problems found in classification trees but at the tradeoff of interpretability.

Tools for implementing random forests as discussed herein are available, by way of non-limiting example, for the statistical software computing language and environment, R. For example, the R package “random Forest,” version 4.6-2, includes tools for creating, processing and utilizing random forests.

### AdaBoost (Adaptive Boosting)

AdaBoost provides a way to classify each of n subjects into two or more categories based on one k-dimensional vector (called a k-tuple) of measurements per subject. AdaBoost takes a series of “weak” classifiers that have poor, though better than random, predictive performance and combines them to create a superior classifier. The weak classifiers that AdaBoost uses are classification and regression trees (CARTs). CARTs recursively partition the dataspace into regions in which all new observations that lie within that region are assigned a certain category label. AdaBoost builds a series of CARTs based on weighted versions of the dataset whose weights depend on the performance of the classifier at the previous iteration. See Han & Kamber (2006) Data Mining, Concepts and Techniques, 2nd Ed. Elsevier: Amsterdam, the content of which is incorporated by reference in its entirety. AdaBoost technically works only when there are two categories to which the observation can belong. For g>2 categories, (g/2) models must be created that classify observations as belonging to a group of not. The results from these models can then be combined to predict the group membership of the particular observation. Predictive performance in this context is defined as the proportion of observations misclassified.

### Convolutional Neural Network

Convolutional Neural Networks (CNNs or ConvNets) are a class of deep, feed-forward artificial neural networks, most commonly applied to analyzing visual imagery. CNNs use a variation of multilayer perceptrons designed to require minimal preprocessing. They are also known as shift invariant or space invariant artificial neural networks (SIANN), based on their shared-weights architecture and translation invariance characteristics. Convolutional networks were inspired by biological processes in that the connectivity pattern between neurons resembles the organization of the animal visual cortex. Individual cortical neurons respond to stimuli only in a restricted region of the visual field known as the receptive field. The receptive fields of different neurons partially overlap such that they cover the entire visual field. CNNs use relatively little pre-processing compared to other image classification algorithms This means that the network learns the filters that in traditional algorithms were hand-engineered. This independence from prior knowledge and human effort in feature design is a major advantage. LeCun and Bengio (1995) “Convolutional networks for images, speech, and time-series,” in Arbib (Ed.), The Handbook of Brain Theory and Neural Networks, MIT Press, the content of which is incorporated by reference in its entirety. Fully convolutional indicates that the neural network is composed of convolutional layers without any fully-connected layers or MLP usually found at the end of the network. Convolutional Neural Network is an example of deep learning.

### Support Vector Machines

Support vector machines (SVMs) are recognized in the art. In general, SVMs provide a model for use in classifying each of n subjects to two or more categories based on one k-dimensional vector (called a k-tuple) per subject. An SVM first transforms the k-tuples using a kernel function into a space of equal or higher dimension. The kernel function projects the data into a space where the categories can be better separated using hyperplanes than would be possible in the original data space. To determine the hyperplanes with which to discriminate between categories, a set of support vectors, which lie closest to the boundary between the disease categories, may be chosen. A hyperplane is then selected by known SVM techniques such that the distance between the support vectors and the hyperplane is maximal within the bounds of a cost function that penalizes incorrect predictions. This hyperplane is the one which optimally separates the data in terms of prediction. Vapnik (1998) Statistical Learning Theory; Vapnik “An overview of statistical learning theory” IEEE Transactions on Neural Networks 10(5): 988-999 (1999) the content of which is incorporated by reference in its entirety. Any new observation is then classified as belonging to any one of the categories of interest, based where the observation lies in relation to the hyperplane. When more than two categories are considered, the process is carried out pairwise for all of the categories and those results combined to create a rule to discriminate between all the categories. See Cristianini, N., & Shawe-Taylor, J. (2000). An Introduction to Support Vector Machines and Other Kernel-based Learning Methods. Cambridge: Cambridge University Press provides some notation for support vector machines, as well as an overview of the method by which they discriminate between observations from multiple groups.

In an exemplary embodiment, a kernel function known as the Gaussian Radial Basis Function (RBF) is used. Vapnik, 1998. The RBF may be used when no a priori knowledge is available with which to choose from a number of other defined kernel functions such as the polynomial or sigmoid kernels. See Han et al. Data Mining: Concepts and Techniques, Morgan Kaufman 3rd Ed. (2012). The RBF projects the original space into a new space of infinite dimension. A discussion of this subject and its implementation in the R statistical language can be found in Karatzoglou et al. “Support Vector Machines in R,” Journal of Statistical Software 15(9) (2006), the content of which is incorporated by reference in its entirety. Other suitable kernel functions include, but are not limited to, linear kernels, radial basis kernels, polynomial kernels, uniform kernels, triangle kernels, Epanechnikov kernels, quartic (biweight) kernels, tricube (triweight) kernels, and cosine kernels. Support vector machines are one out of many possible classifiers that could be used on the data. By way of non-limiting example, and as discussed below, other methods such as naive Bayes classifiers, classification trees, k-nearest neighbor classifiers, etc. may be used on the same data used to train and verify the support vector machine.

### Naïve Bayes Classifier

The set of Bayes Classifiers are a set of classifiers based on Bayes’ Theorem. See, e.g., Joyce (2003), Zalta, Edward N. (ed.), “Bayes’ Theorem”, The Stanford Encyclopaedia of Philosophy (Spring 2019 Ed.), Metaphysics Research Lab, Stanford University, the content of which is incorporated by reference in its entirety.

Classifiers of this type seek to find the probability that an observation belongs to a class given the data for that observation. The class with the highest probability is the one to which each new observation is assigned. Theoretically, Bayes classifiers have the lowest error rates amongst the set of classifiers. In practice, this does not always occur due to violations of the assumptions made about the data when applying a Bayes classifier.

The naïve Bayes classifier is one example of a Bayes classifier. It simplifies the calculations of the probabilities used in classification by making the assumption that each class is independent of the other classes given the data. Naïve Bayes classifiers are used in many prominent anti-spam filters due to the ease of implantation and speed of classification but have the drawback that the assumptions required are rarely met in practice. Tools for implementing naive Bayes classifiers as discussed herein are available for the statistical software computing language and environment, R. For example, the R package “e1071,” version 1.5-25, includes tools for creating, processing and utilizing naive Bayes classifiers.

### Neural Networks

One way to think of a neural network is as a weighted directed graph where the edges and their weights represent the influence each vertex has on the others to which it is connected. There are two parts to a neural network: the input layer (formed by the data) and the output layer (the values, in this case classes, to be predicted). Between the input layer and the output layer is a network of hidden vertices. There may be, depending on the way the neural network is designed, several vertices between the input layer and the output layer. Neural networks are widely used in artificial intelligence and data mining but there is the danger that the models the neural nets produce will over fit the data (i.e., the model will fit the current data very well but will not fit future data well). Tools for implementing neural nets as discussed herein are available for the statistical software computing language and environment, R. For example, the R package “e1071,” version 1.5-25, includes tools for creating, processing and utilizing neural nets.

### k-Nearest Neighbor Classifiers (KNN)

The nearest neighbor classifiers are a subset of memory-based classifiers. These are classifiers that have to “remember” what is in the training set in order to classify a new observation. Nearest neighbor classifiers do not require a model to be fit.

To create a k-nearest neighbor (knn) classifier, the following steps are taken:

1. Calculate the distance from the observation to be classified to each observation in the training set. The distance can be calculated using any valid metric, though Euclidian and Mahalanobis distances are often used. The Mahalanobis distance is a metric that takes into account the covariance between variables in the observations.

2. Count the number of observations amongst the k nearest observations that belong to each group.

3. The group that has the highest count is the group to which the new observation is assigned.

Nearest neighbor algorithms have problems dealing with categorical data due to the requirement that a distance be calculated between two points but that can be overcome by defining a distance arbitrarily between any two groups. This class of algorithm is also sensitive to changes in scale and metric. With these issues in mind, nearest neighbor algorithms can be very powerful, especially in large data sets. Tools for implementing k-nearest neighbor classifiers as discussed herein are available for the statistical software computing language and environment, R. For example, the R package “e1071,” version 1.5-25, includes tools for creating, processing and utilizing k-nearest neighbor classifiers.

In some aspects, the algorithm includes a support vector machine algorithm. Support vector machine algorithms are disclosed in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Duda, Pattern Classification, Second Edition, 2001, John Wiley & Sons, Inc., pp. 259, 262-265; and Hastie, 2001, The Elements of Statistical Learning, Springer, New York; each of which is hereby incorporated by reference in its entirety.

In some aspects, the algorithm may comprise a boosting algorithm. Boosting algorithms are disclosed in Schapire and Freund, 2013, “Boosting: Foundations and Algorithms,” Kybernetes 42(1), pp. 164-166, which is hereby incorporated by reference in its entirety.

In some aspects, the algorithm may comprise any combination of the pattern classification and/or regression algorithms disclosed herein.

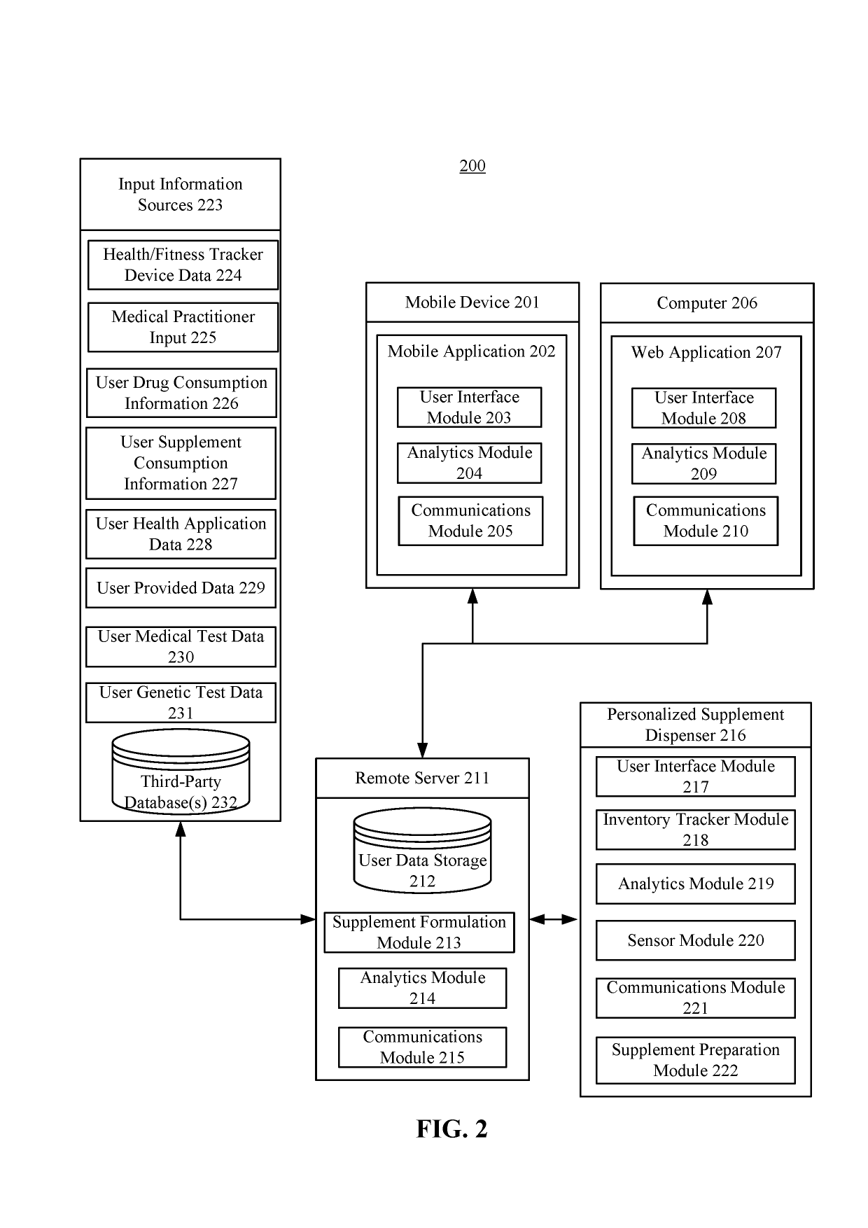

FIG. 2 is a block diagram showing aspects of an exemplary system 200 for generating a personalized dietary supplement according to the present disclosure. This figure highlights potential data sources and the layout of modules used in aspects of the systems described herein.

In this non-limiting example, the system 200 comprises five general components. A remote server 211 is shown, which includes a user data storage 212, a supplement formulation module 213, an analytics module 214, and a communications module 215. The user data storage 212 may store, e.g., any of the user health and/or fitness, or other data, described herein. For example, data collected from one or more wearable devices, sensors, or from the user directly or indirectly, may be stored by the user data storage 212. Note that the presence of user data storage 212 as part of the remote server 211 is an optional configuration; in some aspects, the user data storage may comprise a database or other form of storage hosted separately from the remote server 211. The supplement formulation module 213 of the remote server 211 may comprise executable software code for generating new dietary supplement recipes, or modifying existing recipes, using any of the algorithms, statistical techniques, or AI/ML models describes herein. For example, supplement formulation module 213 may be configured to train and/or perform inference operation using any such AI/ML models, or to perform any algorithms described herein. The supplement formulation module 213 may thus be used to create and modify dietary supplement recipes, and to score or rank candidate dietary supplement recipes (e.g., in order to identify a top-scoring recipe that is best-suited for a user based on a user-selected dietary, health, or fitness goal, or based on any other criteria described herein). The analytics module 214 may comprise executable software code for tracking and/or analyzing user dietary, health, and/or fitness data. For example, the analytics module 214 may be configured to generate charts, graphs, or other text or visual representations based on the user dietary, health, and/or fitness data. For example, the analytics module 214 may track a user’s progress towards a desired health and/or fitness goal, or generate projections based on the user’s current and/or historical biometric, health, fitness, and/or activity data. The output generated by the analytics module 214 may be provided to the user (or to a third party, such a medical or health professional). The analytics module 214 may also be configured to analyze user data and/or data received from the personal supplement dispenser 216 in order to determine or track user preferences, to make recommendations, and/or to automate the ordering of dietary supplement recipe components. For example, the analytics module 214 may analyze the parameters for dietary supplement recipes manually created (or modified) by the user, or the frequency and/or timing of the user generating dietary supplements using the personal supplement dispenser 216, and generate suggestions for the user regarding new dietary supplement components or recipes to consider. The analytics module 214 may also analyze usage data in order to determine when additional dietary supplement components should be ordered (e.g., due to a low quantity or amount in the personal supplement dispenser 216), and may be configured to prompt the user to confirm an order to automatically place an order for the required dietary supplement components. The remote server 211 further includes a communications module 215 that may be used to communicate with the personal supplement dispenser 216, a mobile device 201, a computer 206 and various input information sources 223. In some aspects, the communications module may be configured to communicate with any combination of these system 200 components, via a wireless or wired connection.

As explained above, a supplement dispenser 206 described herein may be operated or controlled by a mobile device 201, or various other input devices (e.g., a tablet, dedicated controller, laptop, desktop, etc.). In this non-limiting example the remote server 211 is configured to communicate with a mobile device 201 and/or a computer 206. As shown by FIG. 2, each of these devices may comprise an application that includes a user interface module, an analytics module, and a communications module. The analytics module and the communications module, respectively, may perform any or all of the functions of the analytics module 214 and the communications module 215 executed on the remote server 211. The user interface module 202/207, may comprise executable software code for generating a GUI that allows the user to interact with and/or control the personal supplement dispenser 216. This GUI may, e.g., allow a user to create, select and/or modify dietary supplement recipes, to input dietary, health and/or fitness information, and to set or select health and/or fitness goals or outcomes, etc. In some aspects, the GUI may allow the user to see the current inventory level of the dietary supplement components in the personal dietary supplement dispenser 216, and/or to order new or additional dietary supplement components for the personal dietary supplement dispenser.

In this example, the remote server 211 is shown in communication with several input information sources 223. A non-limiting set of examples include: health/fitness tracker device data 224, medical practitioner input 225, user drug consumption information 226, user supplement consumption information 227, user health application data 228, user provided data 229, user medical test data 230, user genetic test data 231, and one or more third party databases 232. Health/fitness tracker device data 224 may comprise, e.g., biometric, physiological, and/or activity data obtained using one or more wearable devices, or sensors integrated into or in communication with, devices used by the user (e.g., smart watches, cell phones, dedicated fitness tracker devices). For example, a user’s smart watch may provide heart rate, blood oxygen saturation, or sleep stage/status data for the user. Medical practitioner input 225 may comprise, e.g., recommended amounts, quantities, concentrations, or volumes of one or more dietary supplement components for the user to consume (daily, or according to any other schedule), or one or more target goals for biometric or physiological parameters, provided by a doctor, nurse, dietician, or other medical/health practitioner (or a laboratory directed by any of the foregoing). For example, medical practitioner input 225 may comprise a recommended daily amount of one or more vitamins, probiotics, or other dietary supplement components, or a target weight for the user. User drug consumption information 226, and user supplement consumption information 227, may comprise information about drugs and supplements, respectively, taken by the user. In some cases, a user may set a desired daily minimum or maximum consumption level for a given dietary supplement. Moreover, certain drugs and dietary supplement components may be incompatible or result in adverse effects. Thus, in some aspects it is advantageous for the system 200 to account for other drugs or supplements that a user may consume besides those provided by the personal dietary supplement dispenser 216. User health application data 228 may comprise any biometric, physiological, health and/or fitness parameters, obtained from a third-party application (e.g., separate from the mobile application 202). For example, a user may utilize a fitness tracker device that collects biometric data from the user and provides that data to a third-party. The remote server 211 may be configured to communicate with the third party and obtain third-party application data. User provided data 229 may comprise any information, data, or parameters provided by a user (e.g., biometric, physiological, health and/or fitness parameters, medical history, target health and/or fitness goals, etc.). Various examples of user provided data 229 are described herein and may be accounted for when the server 211 (or any other component of the system 200) generates, modifies, and/or scores personalized dietary supplements, or as part of any analytics functions described herein. User medical test data 230 may comprise, e.g., biometric or physiological data for the user obtained from one or more tests or diagnostic assays (e.g., a bloodwork panel conducted by a laboratory). Similarly, genetic test data 231 may comprise genetic data concerning the presence of absence of specific biomarkers, or DNA sequencing data, for the user. This figure also shows that the remote server 211 may communicate with one or more third-party databases 232. This includes any third-party hosted or controlled servers, databases, or other sources of user data or information. For example, the remote server 211 may be configured to access a third-party database containing current or historical health and/or fitness data for the user.

The system 200 shown in FIG. 2 also includes a personal dietary supplement dispenser 216, which may perform any of the functions of a dietary supplement dispenser described herein. In this case, the personal dietary supplement dispenser 216 includes a user interface module 217, an analytics module 219, and a communications module 221, which may each perform any of the functions provided by the corresponding components in the mobile device 201, the computer 206, and the remote server 211, discussed above. In brief, the user interface module 217 comprises executable software code for generating a GUI that allows a user to control the personal supplement dispenser 216, the analytics module 219 may analyze dietary supplement consumption data, generate suggestions, and/or recommend or place an order when dietary supplement component levels are low.

As discussed above, in some aspects the systems described herein may track the quantity, amount, concentration, and/or volume of the dietary supplement components stored in the personalized dietary supplement 216. In this example, the personal dietary supplement dispenser 216 further comprises an inventory tracker module 218, and a sensor module 220. The sensor module 220 may comprise, e.g., one or more sensors configured to detect and/or measure one or more parameters of the dietary supplement components stored in the personalize dietary supplement dispenser 216 (e.g., one or more scales, each positioned to weight the amount of a different dietary supplement component). In some aspects, the sensor may comprise a mechanical sensor configured to detect a level or volume of a given dietary supplement component, or of a liquid (e.g., water) stored in the personal dietary supplement dispenser 216. Sensor data collected by the one or more sensors of the sensor module 220 may be used by the inventory tracker module 218 to maintain an inventory of the dietary supplement components available in the personal dietary supplement dispenser 216. In some aspects, the sensor module 220 may comprise one or more sensors configured to detect machine-readable labels or codes provided on containers that contain the dietary supplement component. The label or code may, e.g., identify the type and/or source, amount, quantity, volume, and/or expiration date of a dietary supplement component. In such cases, the inventory tracker module 218 may utilize data provided by the machine-readable label or code to determine which dietary supplement components are available to the personal dietary supplement dispenser 216. Moreover, this data may be used by the analytics module 219 (e.g., to track usage, and to provide information such as expiration dates, which are relevant to the determination as to whether to recommend that the user order additional or new dietary supplement components).

The personal dietary supplement dispenser 216 in this example further includes a supplement preparation module 222. This module comprises executable software code for controlling the physical preparation of dietary supplements by the personal dietary supplement dispenser 216. For example, this module may be configured to cause the personal dietary supplement dispenser 216 to combine and/or mix one or more dietary supplement components, and optionally one or more liquids, to produce a personalized dietary supplement for the user based on a recipe received from the remote server 211 or a recipe created or modified by the user (e.g., using the mobile device 201, the computer 206, or via a GUI or physical interface provided by the personal dietary supplement dispenser 216). The supplement preparation module 222 may communicate with any of the other modules (e.g., the inventor tracker module, the analytics module 219, or the communications module 221).

It is envisioned that the functionality provided each of the modules of the system 200 may, in some aspects, be performed by a single module capable of handling multiple functions. Similarly, any of the modules (or functions thereof) may, in some aspects, be performed by a different component of the system 200. For example, in this case the supplement formulation module is part of the remote server 211. In other embodiments, the supplement formulation module may instead be part of the personal dietary supplement dispenser 216. Furthermore, in some aspects a system for preparing personalized dietary supplements may omit any of the modules (or functionality) shown in this example. For example, a personal dietary supplement dispenser 216 may be configured to generate personalized dietary supplements for a user based on manual input (e.g., a user creating or modifying recipes stored in the personal dietary supplement dispenser 216) without the need for a remote server 211. The personal dietary supplement dispenser 216 may be configured to communicate with the input information sources 223, and any other local or remote sources of data or information, without utilizing the remote server 211 as an intermediary.

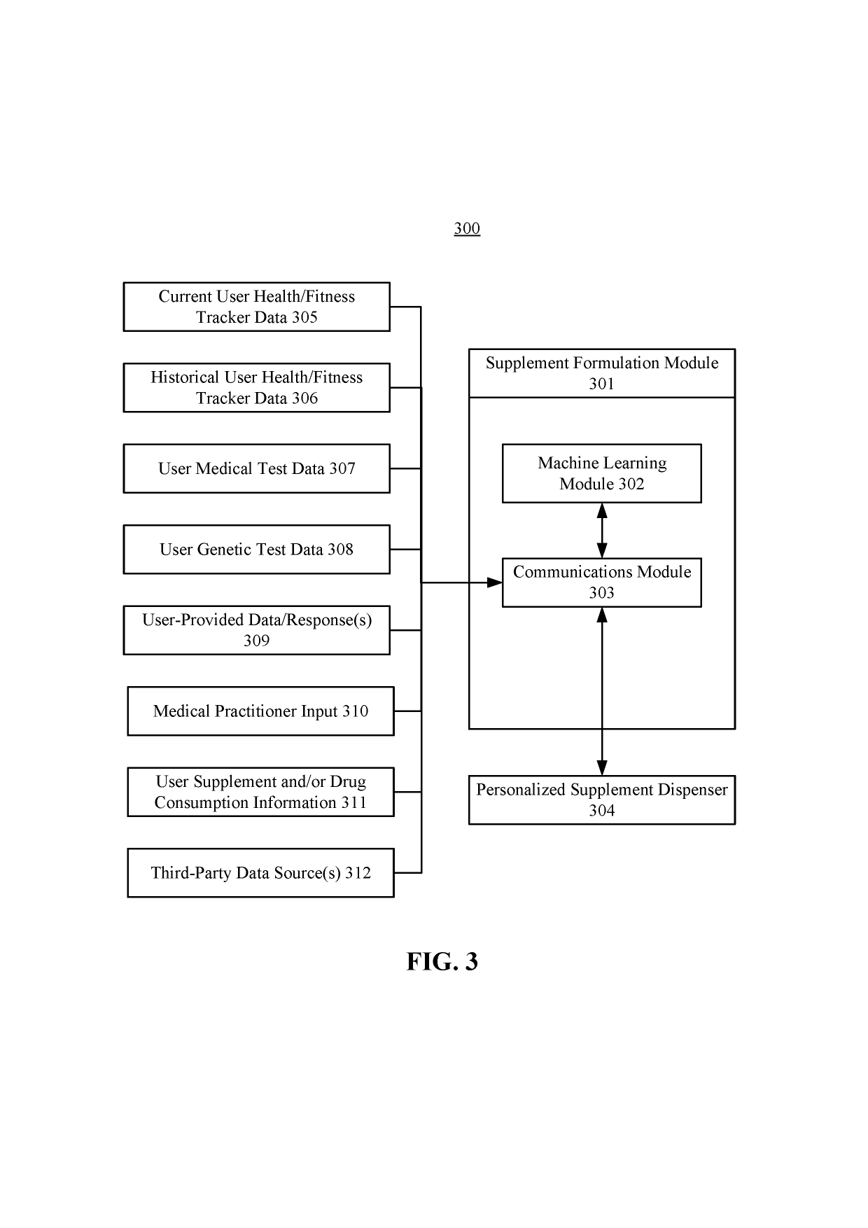

FIG. 3 is a block diagram showing aspects of an exemplary system for generating a personalized dietary supplement according to the present disclosure. This figure highlights potential data sources and the layout of modules used in some aspects of the systems described herein. As discussed above in the context of FIG. 2, a system according to disclosure may utilize various sources of input information (e.g., the sources listed as elements 305-312 in FIG. 3). This information may be obtained from these sources directly or indirectly, and used by a supplement formulation module 301 integrated into a remote server or into a personal dietary supplement dispenser. In this example, the information is obtained using a communications module 303, and provided to machine learning module 302. The machine learning module 302 may perform model training, fine-tuning, or inference using this information as discussed above (e.g., to generate personalized dietary supplement recipes or to score candidate recipes based on any parameters described herein).

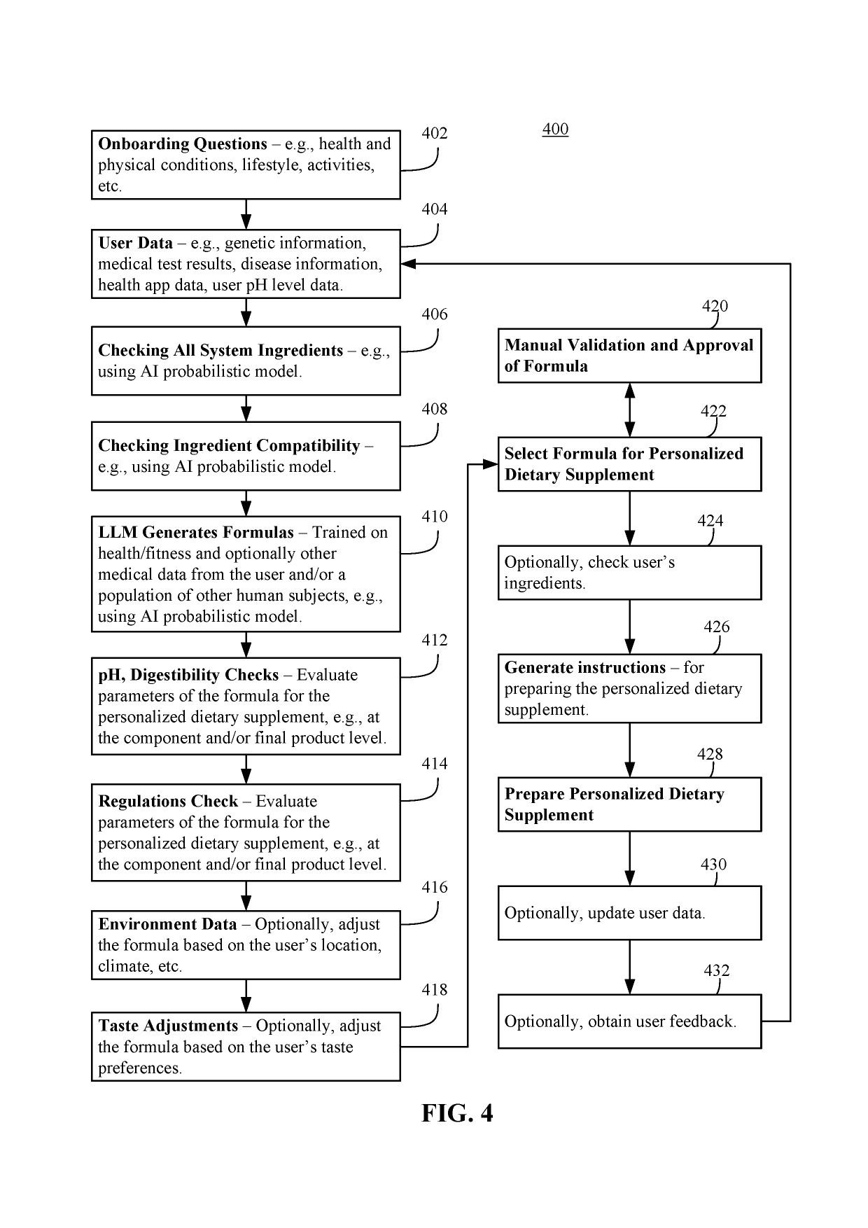

FIG. 4 is a flow chart showing an exemplary method for generating a personalized dietary supplement according to the present disclosure. As illustrated by this figure, an exemplary method for generating a personalized dietary supplement may comprise obtaining input from a user (e.g., via onboarding questions) regarding the user’s health and/or physical conditions, lifestyle, activities, etc. This input may be obtained via any mobile device or computer described herein (e.g., a mobile device 201 or computer 206), or via a remote source (e.g., a remote server 211). Such information may include, e.g., information about the user’s level of fitness, daily activities, inherited or acquired medical conditions or diseases, etc. Optionally, the onboarding questions may collect information about the user’s goals (e.g., weight loss, improved mental wellbeing, improved cardiovascular health, or any other goal described herein). Next, user data is obtained from one or more sources (e.g., as described in the context of FIG. 2 or elsewhere herein). Such data may comprise, e.g., genetic information, medical test results, disease information, data from one or more health-related applications (e.g., a smartphone app) or biometric sensors/devices (e.g., from a smartwatch, or other wearable device comprising one or more biometric or health-related sensors). As shown by this figure, user data may also include pH level data related to one or more tissues, organs, or body surfaces of the user. Next, one or more AI models may be used to check the ingredients (also referred to as “components” herein) available to the system and the compatibility of such ingredients. For example, a pre-trained model may be used to determine whether two or more ingredients stocked by personal dietary supplement dispenser are incompatible, e.g., for health-related reasons, due to taste, texture, or other organoleptic preferences, due to solubility issues, or for any other reason. Next, an LLM or other probabilistic AI model is used to generate one or more dietary supplement formulas. This LLM or other probabilistic AI model may be pre-trained on health/fitness and optionally other medical data from the user and/or from a population of other human subjects (e.g., a population of users of a similar age, gender, weight, or any combination thereof). Next, the generated formulas may be evaluated (e.g., ranked and/or modified) based on one or more parameters (e.g., pH or digestibility) and/or based on compliance with one or more regulations (e.g., target or maximum recommended dosage amounts/levels established by the FDA or any other agency). These evaluations may be performed at the component level and/or at the final product level. For example, the formula as a whole may be evaluated to confirm that the level of a vitamin is below a maximum recommended daily threshold.

Optionally, the process may further include steps wherein environmental data (e.g., the user’s location, climate, etc.) and taste adjustments are considered (e.g., the user’s taste preferences may be used to rank and/or modify the generated formulas). The LLM or other probabilistic AI model may account for either of these parameters when generating the initial formulas. Alternatively, the generated formulas may be modified or optimized in a second round of processing based on either of these parameters.

At the next stage (step 422), a formula for the personalized dietary supplement is selected from the set of one or more previously generated formulas. This selection may comprise one or more rounds of ranking and/or modifying formulas. For example, several initial formulas may be generated, modified and/or ranked based on the user’s environment data and/or taste preferences (as described above), and a final selection may be made based on any combination of criteria described herein. At this point, user input and/or approval may be obtained (e.g., via a GUI of a mobile device application, or on the personal dietary supplement dispenser. This input may validate the automatically generated formula and/or include modifications to the formula. Alternatively, a user may configure a personal dietary supplement dispenser to automatically proceed and generate personalized dietary supplements (e.g., based on a pre-set schedule) without this validation/approval step. When a final recipe is selected (and optionally validated or approved by the user), the following optional step is to check to confirm that the ingredients needed for the selected formula are available (e.g., stocked in the device, available to the user, or orderable from a third party). Next, instructions for preparing the selected formula may be generated (e.g., based on one or more LLMs or probabilistic AI models) and the personalized dietary supplement is prepared by the personal dietary supplement dispenser (step 428). Optionally, user data may be updated and/or feedback may be collected from the user, as shown by this figure.

Any of the functions, including the generative AI aspects, described in the foregoing example may be performed on or by the personal dietary supplement dispenser, on a communicatively linked device or computer (e.g., a user’s mobile device, computer, or other electronic device), or remotely (e.g., by a server or cloud-based computing platform communicatively linked to the personal dietary supplement dispenser or a device or computer capable of communicating with the personal dietary supplement dispenser. Similarly, user input (e.g., regarding the modification of a recipe, or providing health and/or fitness data) may be collected by any means, including via a GUI of the personalized dietary supplement device, or of a communicatively linked electronic device, computer, server, or other platform, either directly or via a third party (e.g., such data may have been initially obtained from the user via a third-party application).

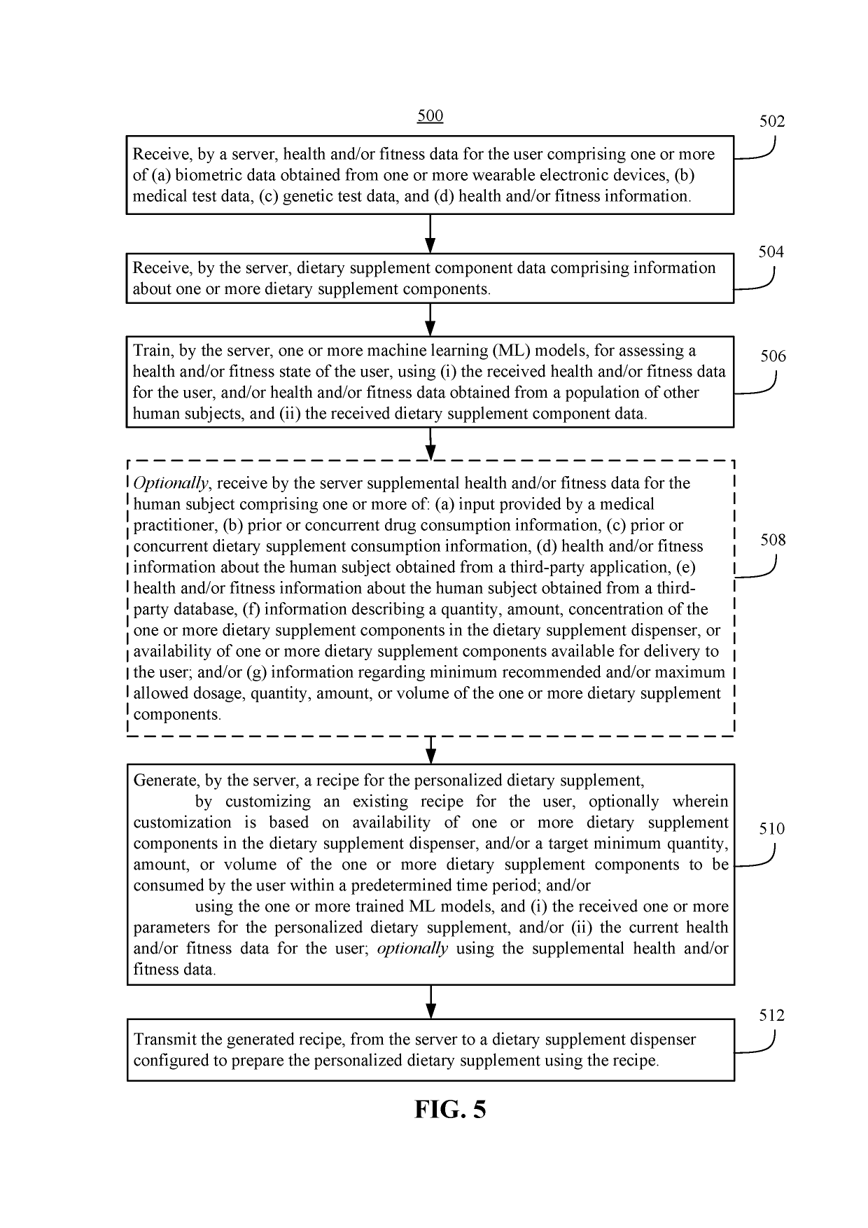

FIG. 5 is a flow chart showing another exemplary method for generating a personalized dietary supplement according to the present disclosure. As illustrated by this figure, an exemplary method for generating a personalized dietary supplement may comprise receiving, by a server, health and/or fitness data for the user comprising one or more of (a) biometric data obtained from one or more wearable electronic devices, (b) medical test data, (c) genetic test data, (d) health and/or fitness information, and/or (e) information describing one or more health and/or fitness goals of the user (402). The server may also receive dietary supplement component data comprising information about one or more dietary supplement components (404). In some aspects, the server may then train one or more machine learning (ML) models, for assessing a health and/or fitness state of the user based at least on the received health and/or fitness data, and the received dietary supplement component data (406). In some aspects, the server may optionally receive supplemental health and/or fitness data for the human subject comprising one or more of: (a) input provided by a medical practitioner, (b) prior or concurrent drug consumption information, (c) prior or concurrent dietary supplement consumption information, (d) health and/or fitness information about the human subject obtained from a third-party application, (e) health and/or fitness information about the human subject obtained from a third-party database, (f) information describing a quantity, amount, concentration of the one or more dietary supplement components in the dietary supplement dispenser, or availability of one or more dietary supplement components available for delivery to the user; and/or (g) information regarding minimum recommended and/or maximum allowed dosage, quantity, amount, or volume of the one or more dietary supplement components (408). In still further aspects, the supplemental health and/or fitness data may comprise any other data or information sources described herein. The server may then proceed to generate a recipe for the personalized dietary supplement, (i) by customizing an existing recipe for the user, optionally wherein customization is based on availability of one or more dietary supplement components in the dietary supplement dispenser, and/or a target minimum quantity, amount, or volume of the one or more dietary supplement components to be consumed by the user within a predetermined time period; and/or (ii) using the one or more trained ML models, and the received one or more parameters for the personalized dietary supplement, and/or the current health and/or fitness data for the user; optionally using the supplemental health and/or fitness data (410). In this example, the generated recipe is then transmitted from the server to a dietary supplement dispenser configured to prepare the personalized dietary supplement using the recipe (e.g., a personal dietary supplement dispenser according to any of the various examples described herein).

As explained above, in some aspects personalized dietary supplement recipes may be generated (or manually created/modified by the user) using an interface of the personal dietary supplement dispenser, or of a mobile device or other controller. Thus, it is understood that the foregoing example is non-limiting and that other methods according to the disclosure may omit the use of a server and instead have some or all of the method steps performed by the personal dietary supplement dispenser (or another component of the system, such as a user’s mobile device or other electronic device that can communicate with the personal dietary supplement dispenser). Moreover, while this example utilized an ML model, as explained herein in some aspects the systems and methods according to the present disclosure may alternatively generate and/or score personalized dietary supplement recipes using algorithms, statistical techniques, and other means unrelated to ML. Indeed, a dietary supplement recipe may be prepared based on a user manually selecting components or modifying an existing recipe, or generated based on dietary requirements or preferences, without any ML training or inference. For example, a personalized dietary supplement recipe may be generated which includes components identified by the user or which are known to improve a health and/or fitness state of the user, or to help users achieve a desired health and/or fitness goal, based on prior research without the use of ML. Thus, alternative methods are contemplated which omit the use of an AI/ML component. Such methods may be advantageous in some contexts, as they require fewer computational and power resources.

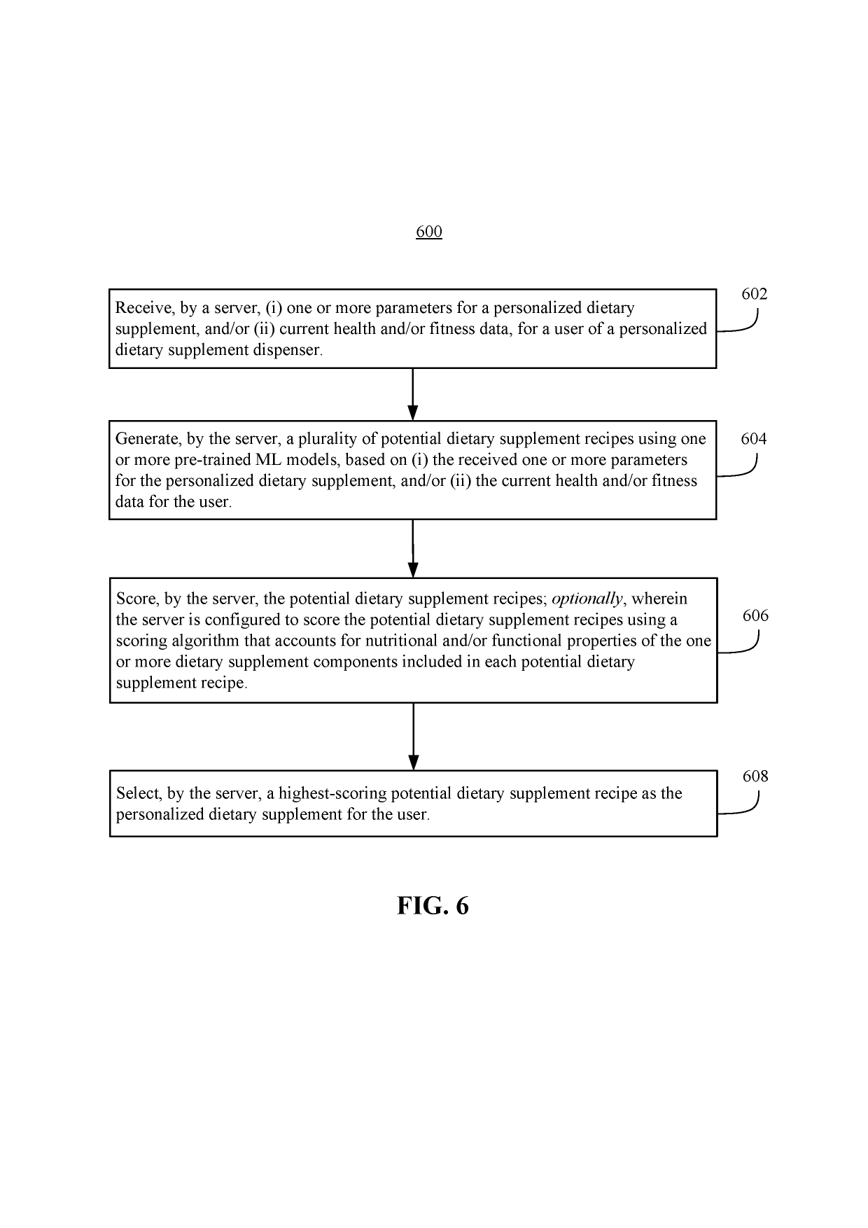

FIG. 6 is a flow chart showing an exemplary method for scoring potential recipes for a personalized dietary supplement according to the present disclosure. As explained above, the systems, methods, and devices described herein may be used to generate personalized dietary supplement recipes (e.g., based on existing templates, manually, or based on user-selected or other parameters). In some aspects, it may be advantageous to generate a plurality of candidate recipes and to score the candidates using AI/ML models, or other algorithms or techniques. For example, an ML model may be trained as discussed above, to identify recipes that are likely to help a user achieve a target health and/or fitness goal, based on historical health, fitness, and dietary data collected from a population of other individuals (e.g., the ML model may be trained to recognize patterns between a user’s supplement consumption and health or fitness-related outcomes). In this figure, an example of such a scoring system is provided. For example, a server may receive (i) one or more parameters for a personalized dietary supplement, and/or (ii) current health and/or fitness data, for a user of a personal dietary supplement dispenser (502). The server may then generate a plurality of potential dietary supplement recipes using one or more pre-trained ML models, based on (i) the received one or more parameters for the personalized dietary supplement, and/or (ii) the current health and/or fitness data for the user (504). Next, the server may score the potential dietary supplement recipes; optionally, wherein the server is configured to score the potential dietary supplement recipes using a scoring algorithm that accounts for nutritional and/or functional properties of the one or more dietary supplement components included in each potential dietary supplement recipe (506). Thereafter, the server may select a highest-scoring potential dietary supplement recipe as the personalized dietary supplement for the user. The selected recipe may then be transmitted to a personal dietary supplement dispenser to be prepared for the user.

This example is non-limiting with respect to the scoring method and the process workflow. In other aspects, other scoring methods may be implemented. For example, recipes may be scored based on their compliance with user preference parameters, based on the health benefits or nutritional value provided by the recipe as a whole, or any other parameters. In some aspects, recipes may be scored based on their compliance or similarity to existing recipes, or recipe guidelines, provided by a third party. For example, recipes may be scored based on compliance to recipe template or guidance provided by a nutritionist, doctor, or any other online or printed source (e.g., recipes may be scored based on compliance with nutritional guidance provided by articles, websites, or other content prepared by a given online “influencer”).

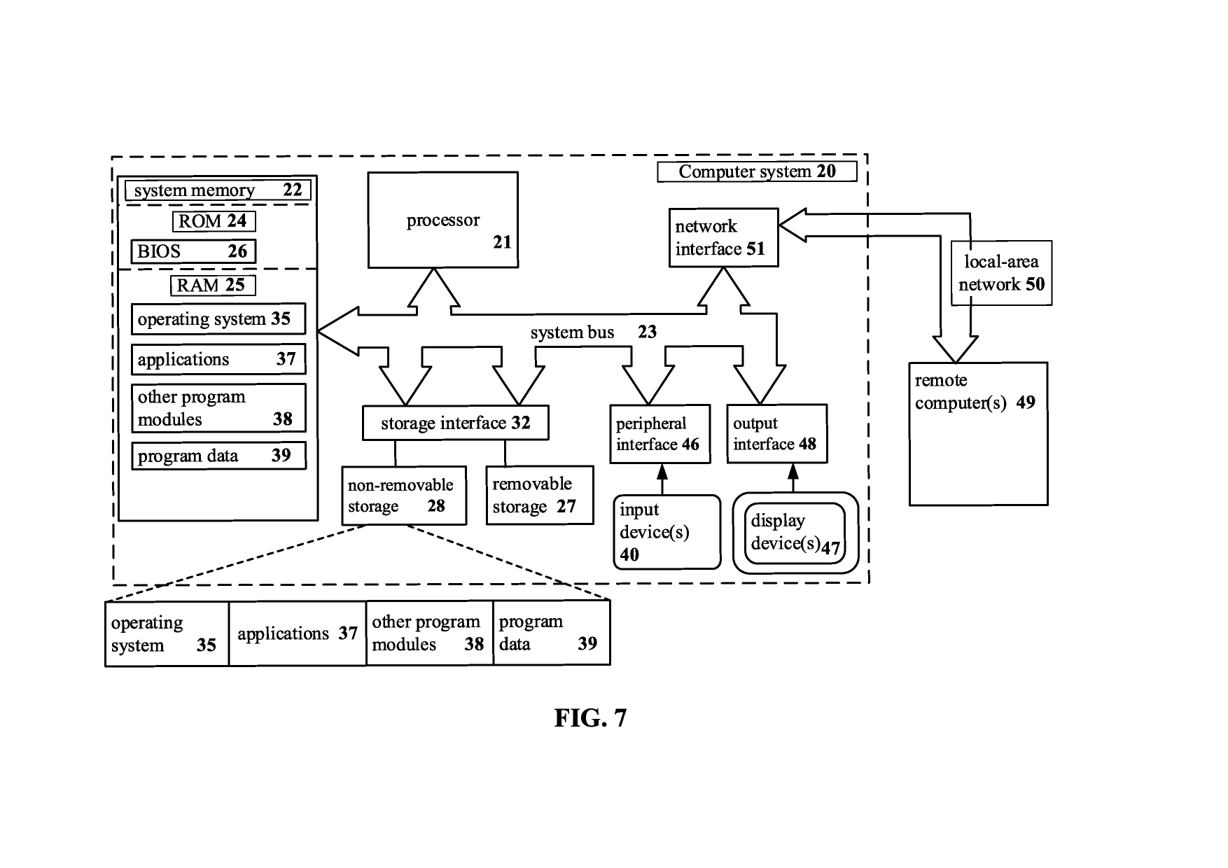

FIG. 7 is a block diagram illustrating a computer system 20 on which aspects of systems and methods disclosed herein may be implemented. The computer system 20 can be in the form of multiple computing devices, or in the form of a single computing device, for example, a desktop computer, a notebook computer, a mobile computing device, a smart phone, a tablet computer, a server, a virtual machine, an embedded device, a consumer electronic device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-6 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term "module" as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module’s functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations, and should not be limited to any particular implementation exemplified herein.

In the interest of clarity, not all of the routine features of the aspects are disclosed herein. It would be appreciated that in the development of any actual implementation of the present disclosure, numerous implementation-specific decisions must be made in order to achieve the developer’s specific goals, and these specific goals will vary for different implementations and different developers. It is understood that such a development effort might be complex and time-consuming, but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A personal dietary supplement dispenser, comprising
a one or more containers, each configured to store one or more dietary supplement ingredients;
one or more reservoirs, each configured to store one or more liquids, powders, or gels;
a communication system;
a controller, configured to control operation of the personal dietary supplement dispenser;
wherein the controller is further configured to
communicate with at least one remote server using the communication system,
generate a personalized dietary supplement for a user, by causing the personal dietary supplement dispenser to combine and/or mix one or more portions of the one or more dietary supplement ingredients, optionally using one or more portions of the one or more liquids, powders, or gels, in response to receiving a recipe for the personalized dietary supplement from the server; and
transmit information about the source, type, status, quantity, and/or amount of the one or more dietary supplement ingredients to the server.

2. The personal dietary supplement dispenser of claim 1, further comprising:
a user interface configured to allow the user to (a) display a recipe generated or received by the personal dietary supplement dispenser, to the user; (b) manually create a recipe for a personalized dietary supplement to prepare; and/or (c) modify one or more parameters of the personalized dietary supplement recipe received from the server.

3. The personal dietary supplement dispenser of claim 1, further comprising:
a) one or more heating or cooling elements, wherein the controller is further configured to heat or cool the personalized dietary supplement using the one or more heating or cooling elements;
b) one or more mixing elements, wherein the controller is further configured to mix one or more of the dietary supplement ingredients and/or the personalized dietary supplement, using the one or more mixing elements; and/or
c) one or more sensors, each capable of detecting one or more signals indicative or a viscosity, temperature, and/or other parameter(s) of the one or more dietary supplement ingredients and/or the personalized dietary supplement.

4. The personal dietary supplement dispenser of claim 1, wherein the controller is further configured to
collect and store usage data for the one or more dietary supplement ingredients and/or the stored liquids, powders, or gels, using one or more sensors, and
transmit the usage data to the remote server using the communication system.

5. The personal dietary supplement dispenser of claim 1, wherein each of the one or more containers includes a machine-readable label comprising information about a source, type, quantity, amount, and/or expiration date, of the dietary supplement ingredient contained in each respective container; and
the controller is configured to cause the personal dietary supplement dispenser to read the machine-readable label using one or more sensors.

6. The personal dietary supplement dispenser of claim 5, wherein the controller is configured to detect when a new container containing a dietary supplement ingredient is added to the one or more containers, and to validate the new container by reading the machine-readable label associated with the new container, and transmit information based on the machine-readable label to the server.

7. The personal dietary supplement dispenser of claim 6, wherein the controller is configured to cause the personal dietary supplement dispenser to use the dietary supplement ingredient stored in the new container only after the new container has been validated.

8. The personal dietary supplement dispenser of claim 1, wherein the controller is further configured to wirelessly communicate with one or more mobile or wearable electronic devices and to allow a user to manually select, create, or modify a personalized dietary supplement recipe using the mobile or wearable electronic device.

9. The personal dietary supplement dispenser of claim 1, wherein the controller is further configured to generate personalized dietary supplements according to a preset schedule and/or based on user proximity data.

10. The personal dietary supplement dispenser of claim 1, wherein the controller is further configured to contact the remote server according to a preset schedule and to download one or more personalized dietary supplement recipes for the user to a local memory of the personal dietary supplement dispenser.

11. The personal dietary supplement dispenser of claim 1, wherein the communication system is a wireless communication system, optionally one that utilizes a near-field communication (NFC) protocol, Bluetooth, or another protocol.

12. The personal dietary supplement dispenser of claim 1, wherein the personal dietary supplement dispenser further comprises one or more sensors, each configured to detect and/or measure at least one parameter of the stored dietary supplement ingredients and/or the stored liquids, powders, or gels.

13. The personal dietary supplement dispenser of claim 1, wherein the server is a smartphone, computer, electronic device, tablet, a remote server, or a cloud-based computing platform.

14. A method for generating a personalized dietary supplement for a user, comprising:
a) providing a personal dietary supplement dispenser comprising
one or more containers, each configured to store one or more dietary supplement ingredients;
one or more reservoirs, each configured to store one or more liquids, powders, or gels;
a communication system; and
a controller, configured to control operation of the personal dietary supplement dispenser and to communicate with at least one server using the communication system;
b) receiving, by the controller, a personalized dietary supplement recipe for the user, from the server; and
c) generating a personalized dietary supplement for the user, using the personal dietary supplement dispenser, by combining and/or mixing one or more portions of the one or more dietary supplement ingredients, optionally using one or more portions of the one or more liquids, powders, or gels, based on the received personalized dietary supplement recipe.

15. The method of claim 14, further comprising
transmitting information about the source, type, status, quantity, amount, and/or expiration date, of the one or more dietary supplement ingredients, from the personal dietary supplement dispenser to the server before or after generating the personalized dietary supplement for the user.

16. The method of claim 14, further comprising:
receiving input from the user, via a user interface of the personal dietary supplement dispenser, wherein the user input modifies one or more parameters of the personalized dietary supplement recipe received from the server.

17. The method of claim 14, further comprising:
a) heating or cooling the personalized dietary supplement using a heating or cooling element integrated into the personal dietary supplement dispenser;
b) mixing one or more of the dietary supplement ingredients and/or the personalized dietary supplement, using one or more mixing elements integrated into the personal dietary supplement dispenser; and/or
c) detecting one or more signals indicative or a viscosity, temperature, and/or other parameter(s) of the one or more dietary supplement ingredients and/or the personalized dietary supplement, using one or more sensors integrated into the personal dietary supplement dispenser.

18. The method of claim 14, wherein the controller is further configured to
collect and store usage data for the one or more dietary supplement ingredients and/or the stored liquids, powders, or gels, using one or more sensors, and
transmit the usage data to the server using the communication system.

19. The method of claim 14, wherein each of the one or more containers includes a machine-readable label comprising information about the source, type, quantity, amount, or expiration date, of the dietary supplement ingredients contained in each respective container; and
the controller is configured to cause the personal dietary supplement dispenser to read the machine-readable label using one or more sensors.

20. The method of claim 19, wherein the controller is configured to detect when a new container containing a dietary supplement ingredient is added to the one or more containers, and to validate the new container by reading the machine-readable label associated with the new container, and transmit information based on the machine-readable label to the server.
